SEQUENCE LISTING (23 sequences)

SEQ ID NO: 1 - DNA, 1959 bp, Viral
atgcatcccacgttaagccggcgccacctcccgattcggggtggaaagccgaaaagactgaaaatccccttaagcttcgcctccatcgcgtggttccttactctgtcaataactcctcaagttaatggtaaacgccttgtgaacagtccgaactcccataaacccttatctctcacctggttacttactgactccggtacaggtattaatattaacagcactcaaggggaggctcccttggggacctggtggcctgaattatatgtctgccttcgatcagtaatccctggtctcaatgaccaggccacaccccccgatgtactccgtgcttacgggttttacgtttgcccaggacccccaaataatgaagaatattgtggaaatcctcaggatttcttttgcaagcaatggagctgcgtaacttctaatgatgggaattggaaatggccagtctctcagcaagacagagtaagttactcttttgttaacaatcctaccagttataatcaatttaattatggccatgggagatggaaagattggcaacagcgggtacaaaaagatgtacgaaataagcaaataagctgtcattcgttagacctagattacttaaaaataagtttcactgaaaaaggaaaacaagaaaatatcctaaaatgggtacatggtatgtcttggggaatggtatattatggaggctcgggtaaacaaccaggctccattctaactattcgcctcaaaataaaccagctggagcctccaatggctataggaccaaatacggtcttgacgggtcaaagacccccaacccaaggaccaggaacatcctctaacataacttctggatcagaccccactgagtctaacagcacgactaaaatgggggcaaaactttttagcctcatccagggagcttttcaagctcttaactccacgactccagaggctacctcttcttgttggctatgcttagctttgggcccaccttactatgaaggaatggctagaagagggaaattcaatgtgacaaaagaacatagagaccaatgcacatggggatcccaaaataagcttacccttactgaggtttctggaaaaggcacctgcataggaaaggttcccccatcccaccaacacctttgtaaccacactgaagcctttaatcaaacctctgagagtcaatatctggtacctggttatgacaggtggtgggcatgtaatactggattaaccccttgtgtttccactttggtttttaaccaaactaaagatttttgcattatggtccaaattgttccccgagtgtattactatcccgaaaaagcaatccttgatgaatatgactacagaaatcatcgacaaaagagagaacccatatctctgacacttgctgtgatgctcggacttggagtggcagcaggtgtaggaacaggaacagctgccctggtcacgggaccacagcagctagaaacaggacttagtaacctacatcgaattgtaacagaagatctccaagccctagaaaaatctgtcagtaacctggaggaatccctaacctccttatctgaagtagtcctacagaatagaagagggttagatttattatttctaaaagaaggaggattatgtgtagccttgaaggaggaatgctgtttttatgtggatcattcaggggccatcagagactccatgagcaagcttagagaaaggttggagaagcgtcgaagggaaaaggaaactactcaagggtggtttgagggatggttcaacaggtctctttggttggctaccctactttctgctttaacaggacccttaatagtcctcctcctgttactcacagttgggccatgtattattaacaagttaattgccttcattagagaacgaataagtgcagtccagatcatggtacttagacaacagtaccaaagcccgtctagcagggaagctggccgc

SEQ ID NO: 2 - protein, 653 aa, Viral
MHPTLSRRHLPIRGGKPKRLKIPLSFASIAWFLTLSITPQVNGKRLVNSPNSHKPLSLTWLLTDSGTGININSTQGEAPLGTWWPELYVCLRSVIPGLNDQATPPDVLRAYGFYVCPGPPNNEEYCGNPQDFFCKQWSCVTSNDGNWKWPVSQQDRVSYSFVNNPTSYNQFNYGHGRWKDWQQRVQKDVRNKQISCHSLDLDYLKISFTEKGKQENILKWVHGMSWGMVYYGGSGKQPGSILTIRLKINQLEPPMAIGPNTVLTGQRPPTQGPGTSSNITSGSDPTESNSTTKMGAKLFSLIQGAFQALNSTTPEATSSCWLCLALGPPYYEGMARRGKFNVTKEHRDQCTWGSQNKLTLTEVSGKGTCIGKVPPSHQHLCNHTEAFNQTSESQYLVPGYDRWWACNTGLTPCVSTLVFNQTKDFCIMVQIVPRVYYYPEKAILDEYDYRNHRQKREPISLTLAVMLGLGVAAGVGTGTAALVTGPQQLETGLSNLHRIVTEDLQALEKSVSNLEESLTSLSEVVLQNRRGLDLLFLKEGGLCVALKEECCFYVDHSGAIRDSMSKLRERLEKRRREKETTQGWFEGWFNRSLWLATLLSALTGPLIVLLLLLTVGPCIINKLIAFIRERISAVQIMVLRQQYQSPSSREAGR

SEQ ID NO: 3 - DNA, 1959 bp, Viral
atgcatcccacgttaagccggcgccacctcccgattcggggtggaaagccgaaaagactgaaaatccccttaagcttcgcctccatcgcgtggttccttactctgtcaataactcctcaagttaatggtaaacgccttgtggacagcccgaactcccataaacccttatctctcacctggttacttactgactccggtacaggtattaatattaacagcactcaaggggaggctcccttggggacctggtggcctgaattatatgtctgccttcgatcagtaatccctggtctcaatgaccaggccacaccccccgatgtactccgtgcttacgggttttacgtttgcccaggacccccaaataatgaagaatattgtggaaatcctcaggatttcttttgcaagcaatggagctgcgtaacttctaatgatgggaattggaaatggccagtctctcagcaagacagagtaagttactcttttgttaacaatcctaccagttataatcaatttaattatggccatgggagatggaaagattggcaacagcgggtacaaaaagatgtacgaaataagcaaataagctgtcattcgttagacctagattacttaaaaataagtttcactgagaaaggaaaacaagaaaatatcctaaaatgggtaaatggtatgtcttggggaatggtatattatggaggctcgggtaaacaaccaggctccattctaactattcgcctcaaaataaaccagctggagcctccaatggctataggaccaaatacggtcttgacgggtcaaagacccccaacccaaggaccaggaccatcctctaacataacttctggatcagaccccactgagtctaacagcacgactaaaatgggggcaaaactttttagcctcatccagggagcttttcaagctcttaactccacgactccagaggctacctcttcttgttggctatgcttagctttgggcccaccttactatgaaggaatggctagaagagggaaattcaatgtgacaaaaggacatagagacccatgcacatggggatcccaaaataagcttacccttactgaggtttttggaaaaggcacctgcataggaaaggttcccccatcccaccaacacctttgtaaccacactgaagcctttaatcgaacctctgagagtcagtatctggtacctggttatgacaggtggtgggcatgtaatactggattaaccccttgtgtttccactttggtttttaaccaaactaaagatttttgcattatggtccaaattgttccccgagtgtattactatcccgaaaaagcaatccttgatgaatatgactacagaaatcatcgacaaaagagagaacccatatctctgacacttgctgtgatgctcggacttggagtggcagcaggtgtaggaacaggaacagctgccctggtcacgggaccacagcagctagaaacaggacttagtaacctacatcgaattgtaacagaagatctccaagccctagaaaaatctgtcagtaacctggaggaatccctaacctccttatctgaagtagtcctacagaatagaagagggttagatttattatttctaaaagaaggaggattatgtgtagccttgaaggaggaatgctgtttttatgtggatcattcaggggccatcagagactccatgagcaagcttagagaaaggttggagaagcgtcgaagggaaaaggaaactactcaagggtggtttgagggatggttcaacaggtctccttggttggctaccctactttctgctttaacaggacccttaatagtcctcctcctgttactcacagttgggccatgtattattaacaagttaattgccttcattagagaacgaataagtgcagtccagatcatggtacttagacaacagtaccaaagcccgtctagcagggaagctggccgc

SEQ ID NO: 4 - protein, 653 aa, Viral
MHPTLSRRHLPIRGGKPKRLKIPLSFASIAWFLTLSITPQVNGKRLVDSPNSHKPLSLTWLLTDSGTGININSTQGEAPLGTWWPELYVCLRSVIPGLNDQATPPDVLRAYGFYVCPGPPNNEEYCGNPQDFFCKQWSCVTSNDGNWKWPVSQQDRVSYSFVNNPTSYNQFNYGHGRWKDWQQRVQKDVRNKQISCHSLDLDYLKISFTEKGKQENILKWVNGMSWGMVYYGGSGKQPGSILTIRLKINQLEPPMAIGPNTVLTGQRPPTQGPGPSSNITSGSDPTESNSTTKMGAKLFSLIQGAFQALNSTTPEATSSCWLCLALGPPYYEGMARRGKFNVTKGHRDPCTWGSQNKLTLTEVFGKGTCIGKVPPSHQHLCNHTEAFNRTSESQYLVPGYDRWWACNTGLTPCVSTLVFNQTKDFCIMVQIVPRVYYYPEKAILDEYDYRNHRQKREPISLTLAVMLGLGVAAGVGTGTAALVTGPQQLETGLSNLHRIVTEDLQALEKSVSNLEESLTSLSEVVLQNRRGLDLLFLKEGGLCVALKEECCFYVDHSGAIRDSMSKLRERLEKRRREKETTQGWFEGWFNRSPWLATLLSALTGPLIVLLLLLTVGPCIINKLIAFIRERISAVQIMVLRQQYQSPSSREAGR

SEQ ID NO: 5 - DNA, 20 bp, Artificial Sequence
  PCR Primer Oligonucleotide
agccctcactccttctctag

SEQ ID NO: 6 - DNA, 18 bp, Artificial Sequence
  PCR Primer Oligonucleotide
gatgtttggccgaggcgg

SEQ ID NO: 7 - DNA, 22 bp, Artificial Sequence
  PCR Primer Oligonucleotide
ccaaagcatctttggacctacc

SEQ ID NO: 8 - DNA, 20 bp, Artificial Sequence
  PCR Primer Oligonucleotide
tcacgatgaagacaggtggg

SEQ ID NO: 9 - DNA, 1853 bp, Homo sapiens
ggcacgagggtccctgggccggacggcggtgtcccggcgtggcgggaagccggcactggagcgggagcgcactgggcgcgggaccgggaggcgcagggaccggacggctcccgagtcgcccacctgacgctagaagaagtcttcacttcccaggagagccaaagcgtgtctggccctaggtgggaaaagaactggctgtgacctttgccctgacctggaagggcccagccttgggctgaatggcagcacccacgcccgcccgtccggtgctgacccacctgctggtggctctcttcggcatgggctcctgggctgcggtcaatgggatctgggtggagctacctgtggtggtcaaagagcttccagagggttggagcctcccctcttacgtctctgtgcttgtggctctggggaacctgggtctgctggtggtgaccctctggaggaggctggccccaggaaaggacgagcaggtccccatccgggtggtgcaggtgctgggcatggtgggcacagccctgctggcctctctgtggcaccatgtggccccagtggcaggacagttgcattctgtggccttcttagcactggcctttgtgctggcactggcatgctgtgcctcgaatgtcactttcctgcccttcttgagccacctgccacctcgcttcttacggtcattcttcctgggtcaaggcctgagtgccctgctgccctgcgtgctggccctagtgcagggtgtgggccgcctcgagtgcccgccagcccccatcaacggcacccctggccccccgctcgacttccttgagcgttttcccgccagcaccttcttctgggcactgactgcccttctggtcgcttcagctgctgccttccagggtcttctgctgctgttgccgccaccaccatctgtacccacaggggagttaggatcaggcctccaggtgggagccccaggagcagaggaagaggtggaagagtcctcaccactgcaagagccaccaagccaggcagcaggcaccacccctggtccagaccctaaggcctatcagcttctatcagcccgcagtgcctgcctgctgggcctgttggccgccaccaacgcgctgaccaatggcgtgctgcctgccgtgcagagcttttcctgcttaccctacgggcgtctggcctaccacctggctgtggtgctgggcagtgctgccaatcccctggcctgcttcctggccatgggtgtgctgtgcaggtccttggcagggctgggcggcctctctctgctgggcgtgttctgtgggggctacctgatggcgctggcagtcctgagcccctgcccgcccctggtgggcacctcggcgggggtggtcctcgtggtgctgtcgtgggtgctgtgtcttggcgtgttctcctacgtgaaggtggcagccagctccctgctgcatggcgggggccggccggcattgctggcagccggcgtggccatccaggtgggctctctgctcggcgctgttgctatgttccccccgaccagcatctatcacgtgttccacagcagaaaggactgtgcagacccctgtgactcctgagcctgggcaggtggggaccccgctccccaacacctgtctttccctcaatgctgccaccatgcctgagtgcctgcagcccaggaggcccgcacaccggtacactcgtggacacctacacactccataggagatcctggctttccagggtgggcaagggcaaggagcaggcttggagccagggaccagtgggggctgtagggtaagcccctgagcctgggacctacatgtggtttgcgtaataaaacatttgtatttaaaaaaaaaaaaaaaaaaaaa

SEQ ID NO: 10 - protein, 445 aa, Homo sapiens
MAAPTPARPVLTHLLVALFGMGSWAAVNGIWVELPVVVKELPEGWSLPSYVSVLVALGNLGLLVVTLWRRLAPGKDEQVPIRVVQVLGMVGTALLASLWHHVAPVAGQLHSVAFLALAFVLALACCASNVTFLPFLSHLPPRFLRSFFLGQGLSALLPCVLALVQGVGRLECPPAPINGTPGPPLDFLERFPASTFFWALTALLVASAAAFQGLLLLLPPPPSVPTGELGSGLQVGAPGAEEEVEESSPLQEPPSQAAGTTPGPDPKAYQLLSARSACLLGLLAATNALTNGVLPAVQSFSCLPYGRLAYHLAVVLGSAANPLACFLAMGVLCRSLAGLGGLSLLGVFCGGYLMALAVLSPCPPLVGTSAGVVLVVLSWVLCLGVFSYVKVAASSLLHGGGRPALLAAGVAIQVGSLLGAVAMFPPTSIYHVFHSRKDCADPCDS

SEQ ID NO: 11 - DNA, 1732 bp, Homo sapiens
gctagaagaagtcttcacttcccaggagagccaaagcgtgtctggccctaggtgggaaaagaactggctgtgacctttgccctgacctggaagggcccagccttgggctgaatggcagcacccacgcccgcccgtccggtgctgacccacctgctggtggctctcttcggcatgggctcctgggctgcggtcaatgggatctgggtggagctacctgtggtggtcaaagagcttccagagggttggagcctcccctcttacgtctctgtgcttgtggctctggggaacctgggtctgctggtggtgaccctctggaggaggctggccccaggaaaggacgagcaggtccccatccgggtggtgcaggtgctgggcatggtgggcacagccctgctggcctctctgtggcaccatgtggccccagtggcaggacagttgcattctgtggccttcttagcactggcctttgtgctggcactggcatgctgtgcctcgaatgtcactttcctgcccttcttgagccacctgccacctcgcttcttacggtcattcttcctgggtcaaggcctgagtgccctgctgccctgcgtgctggccctagtgcagggtgtgggccgcctcgagtgcccgccagcccccatcaacggcacccctggccccccgctcgacttccttgagcgttttcccgccagcaccttcttctgggcactgactgcccttctggtcgcttcagctgctgccttccagggtcttctgctgctgttgccgccaccaccatctgtacccacaggggagttaggatcaggcctccaggtgggagccccaggagcagaggaagaggtggaagagtcctcaccactgcaagagccaccaagccaggcagcaggcaccacccctggtccagaccctaaggcctatcagcttctatcagcccgcagtgcctgcctgctgggcctgttggccgccaccaacgcgctgaccaatggcgtgctgcctgccgtgcagagcttttcctgcttaccctacgggcgtctggcctaccacctggctgtggtgctgggcagtgctgccaatcccctggcctgcttcctggccatgggtgtgctgtgcaggtccttggcagggctgggcagcctctctctgctgggcgtgttctgtgggggctacctgatggcgctggcagtcctgagcccctgcccgcccctggtgggcacctcggcgggggtggtcctcgtggtgctgtcgtgggtgctgtgtcttggcgtgttctcctacgtgaaggtggcagccagctccctgctgcatggcgggggccggccggcattgctggcagccggcgtggccatccaggtgggctctctgctcggcgctgttgctatgttccccccgaccagcatctatcacgtgttccacagcagaaaggactgtgcagacccctgtgactcctgagcctgggcaggtggggaccccgctccccaacacctgtctttccctcaatgctgccaccatgcctgagtgcctgcagcccaggaggcccgcacaccggtacactcgtggacacctacacactccataggagatcctggctttccagggtgggcaagggcaaggagcaggcttggagccagggaccagtgggggctgtagggtaagcccctgagcctgggacctacatgtggtttgcgtaataaaacatttatatttaaaaaaaaaaaaaaaaaaaaaaaaaaaa

SEQ ID NO: 12 - protein, 445 aa, Homo sapiens [sequence omitted]

SEQ ID NO: 13 - DNA, 1473 bp, Homo sapiens
aaagcatctttggacctacctagggaaggacctgcctgtgacctttgccctgtcctggagggtccagctttgggctgaatggcagcacccacgctgggccgtctggtgctgacccacctgctggtggccctttttggcatgggctcctgggctgctgtgaacgggatctgggtggagctgcctgtggtggtaaaagaccttccagagggttggagcctcccctcatacctctctgtggttgtggcgctgggaaacctgggtctgctggtggtgaccctgtggaggcggctggccccgggcaagggcgagcaggtccccatccaggtggtacaggtgctgagtgtagtgggcacagccctgctggcccctctgtggcaccacgtggccccagtggcagggcagctccactctgtggccttcctaactctggccttggtgttggcaatggcctgttgtacctctaatgtcactttcctgcccttcctgagccacctgccacctcctttcttacggtctttcttcctgggtcagggtctcagtgccctactcccctgtgtgctggccctagtgcaaggtgtgggccgcctcgagtgcccaccagcgcccaccaatggcacctctgggcctcccctcgacttccctgagcgttttcctgccagcaccttcttctgggcactgactgcccttctggtcacttcagctgccgccttccggggtctcctgttgctgttgccatcactaccctctgtaaccacagggggctcagggcctgaacttcaactgggatccccaggagcagaggaggaagagaaggaggaagaagaggctttgccattgcaggagccaccgagccaggcagcaggcaccatccctggcccagaccctgaggcccatcagctgttctcagcccatggtgccttcctgctgggcctgatggccttcaccagtgccgtgaccaatggcatgctgccttctgtgcagagcttttcctgtttgccctatgggcgcctggcctaccacctggctgtggtgctgggcagtgccgccaacccccttgcctgcttcctggccatgggcgtgctgtgcaggtccctggcagggctggttggtctttctctgctgggcatgctctttggggcctacctgatggcactggcaatcctgagcccctgcccacccctggtgggcaccactgcaggggtggtccttgtggtgctgtcgtgggtgctgtgtctgtgtgtgttctcatatgtgaaggtggctgcaagctccctgctgcatggtgggggtcggccggcattgctggcagctggtgtggccatccaagtgggctccctgcttggtgccggtgccatgttccctcccaccagcatctaccacgtgtttcaaagcagaaaggactgtgtagacccctgtggcccctgagcctgggcaggtggggacccaactccaccccacctgtcttcatcgtga

SEQ ID NO: 14 - protein, 448 aa, Homo sapiens [sequence omitted]

SEQ ID NO: 15 - DNA, 1347 bp, Baboon
atggcagcacccacgctgggccatctggtgctgacccacctgctggtggcccttctcggcatgggctcctgggctgctgtcaacggcatctgggtggagctacctgtggtggtaaaacaccttccagagggttggagcctcccctcatacctctctgtggttgtggcactgggaaacctgggtctgctggtggtgactctgtggaggcggctggccccgggcaagggcgagcgggtccccatccaggtggtacaggtgctgagtgtagtgggcacagccctgctggcccctctgtggcaccacgtggccccagtggcagggcaactccactccgtggccttcctaacactggccttagtgttggcactggcctgctgtacctctaatgtcactttcctgcccttcctgagccacctgccacctcctttcttacggtctttcttcctgggtcagggtctcagtgccctgctcccctgtgtgctagccctagtgcagggtgtaggccgcctcgagtgctcgccagcgcccaccaatggcacctcagggcctcccctcaacttccctgagcgttttcctgccagcaccttctactgggcactgactgcccttctggtcacttcggctgccgccttccagggtctcctgttgctgttgccatcactaccatctgtaaccacagggggcgcagggcctgaacttccactgggatccccaggagcagaggaggaagagaaggaggaagaagaggctttgccattgcaggagccaccaagccaggcagcaggcaccatccctggcccagaccctgaggcccatcagctgttctcagcccatggtgccttcctgctgggcctgctggccatcaccagtgccctgaccaatggcgtgctgcctgccgtgcagagcttttcctgtttgccctatgggcgcttggcctaccacctggctgtggtgctgggcagtgccgccaacccccttgcctgcttcctggccatgggcgtgctgtgcaggtccctggcagggctggttggtctttctctgctgggcatgctctttggggcctacctgatggtactggcaatcctgagcccctgcccacccctggtgggcaccaccgcaggggtggtccttgtggtactgtcgtgggtgctgtgtctttgtgtgttctcatacgtgaaggtggctgcaagctccctgctgcatggtgggggtcggccggcattgctggcggctggtgtggccatccagatgggctccctgcttggtgccggcaccatgttccctcctaccagcatctaccacgtgtttcaaagcagaaaggactgtgtagacccctgtggcccctga

SEQ ID NO: 16 - protein, 448 aa, Baboon
MAAPTLGHLVLTHLLVALLGMGSWAAVNGIWVELPVVVKHLPEGWSLPSYLSVVVALGNLGLLVVTLWRRLAPGKGERVPIQVVQVLSVVGTALLAPLWHHVAPVAGQLHSVAFLTLALVLALACCTSNVTFLPFLSHLPPPFLRSFFLGQGLSALLPCVLALVQGVGRLECSPAPTNGTSGPPLNFPERFPASTFYWALTALLVTSAAAFQGLLLLLPSLPSVTTGGAGPELPLGSPGAEEEEKEEEEALPLQEPPSQAAGTIPGPDPEAHQLFSAHGAFLLGLLAITSALTNGVLPAVQSFSCLPYGRLAYHLAVVLGSAANPLACFLAMGVLCRSLAGLVGLSLLGMLFGAYLMVLAILSPCPPLVGTTAGVVLVVLSWVLCLCVFSYVKVAASSLLHGGGRPALLAAGVAIQMGSLLGAGTMFPPTSIYHVFQSRKDCVDPCGP

SEQ ID NO: 17 - protein, 445 aa, Artificial Sequence
  Consensus sequence of SEQ ID NO 12, 14 and 16.
MAAPTLGRLVLTHLLVALFGMGSWAAVNGIWVELPVVVKLPEGWSLPSYLSVVVALGNLGLLVVTLWRRLAPGKGEQVPIQVVQVLSVVGTALLAPLWHHVAPVAGQLHSVAFLTLALVLALACCTSNVTFLPFLSHLPPPFLRSFFLGQGLSALLPCVLALVQGVGRLECPPAPTNGTSGPPLDFPERFPASTFFWALTALLVTSAAAFQGLLLLLPSLPSVTTGGGPELQLGSPGAEEEEKEEEEALPLQEPPSQAAGTIPGPDPEAHQLFSAHGAFLLGLLATSALTNGVLPAVQSFSCLPYGRLAYHLAVVLGSAANPLACFLAMGVLCRSLAGLVGLSLLGMLFGAYLMALAILSPCPPLVGTTAGVVLVVLSWVLCLCVFSYVKVAASSLLHGGGRPALLAAGVAIQVGSLLGAGAMFPPTSIYHVFQSRKDCVDPCGP

SEQ ID NO: 18 - DNA, 19 bp, Artificial Sequence
  PCR Primer Oligonucleotide
cccagtggcaggacagttg

SEQ ID NO: 19 - DNA, 16 bp, Artificial Sequence
  PCR Primer Oligonucleotide
tcagcgcgttggtggc

SEQ ID NO: 20 - DNA, 20 bp, Artificial Sequence
  PCR Primer Oligonucleotide
gtkaccttygcyykwcctgg

SEQ ID NO: 21 - DNA, 19 bp, Artificial Sequence
  PCR Primer Oligonucleotide
ggagykgggtccccacctg

SEQ ID NO: 22 - DNA, 17 bp, Artificial Sequence
  PCR Primer Oligonucleotide
aatggcagcaccymcgc

SEQ ID NO: 23 - DNA, 18 bp, Artificial Sequence
  PCR Primer Oligonucleotide
tcaggggccacaggggtc

(19) United States
(12) Patent Application Publication
(10) Pub. No.: US 2003/0175904 A1
(43) Pub. Date: Sep. 18, 2003

(54) Molecular sequence of pig endogenous retrovirus receptors and methods of use

(76) Inventors: Clive Patience, Beverly, MA (US); Thomas Ericsson, Newtonville, MA (US); Beth A. Oldmixon, Allston, MA (US)

Correspondence Address: Alan J. Grant, Esq., c/o Carella, Byrne, Bain, Gilfillan,, Cecchi, Stewart & Olstein, 6 Becker Farm Road, Roseland, NJ, 07068, US

(21) Appl. No.: 10/029,656

(22) Filed: Dec. 21, 2001

Related U.S. Application Data
(60) Provisional application No. 60/285,103, filed on Apr. 20, 2001.

(51) Int. Cl. C12Q 1/70; G01N 33/53; C12P 19/34; A61K 39/12; A61K 39/21; C12N 5/06; C12N 5/16

(52) U.S. Cl. 435/91.1; 435/91.33; 424/204.1; 424/207.1; 435/5; 435/345; 435/7.1

(57) ABSTRACT

Polypeptide sequences for PERV-A receptors found on primate cells, including human, and the polynucleotides encoding those polypeptide receptor sequences are disclosed along with processes for using the PERV-A receptors in both in vitro and in vivo screening assays and models for agents that block viral infection. Also disclosed are processes for detecting the presence of PERV receptors in a tissue, blocking virus binding to receptors and protection of an animal, such as a human patient, from PERV infection. Antibodies that react with PERV receptors, vectors comprising the polynucleotides encoding such receptors and recombinant cells that express these receptors are also described.

This application claims priority of U.S. Provisional Application Serial No. 60/285,103, filed Apr. 20, 2001, the disclosure of which is hereby incorporated by reference in its entirety.

### FIELD OF THE INVENTION

This invention relates generally to porcine endogenous retroviral receptors and methods of using them in the detection of tissues infectable by PERV and in the inhibition of infection of such tissues, especially for xenotransplantation.

### BACKGROUND OF THE INVENTION

Organ procurement currently poses one of the major problems in solid organ transplantation, since the number of patients requiring transplants far exceeds the number of organs available. A means of eliminating the shortage of donor organs for transplantation is to develop technologies to transplant non-human organs into humans, i.e., xenotransplantation. However, a central concern regarding xenotransplantation is the risk of zoonosis, infection by organisms transferred with the xenograft into both the transplant recipient and the general population, including “emerging infections” caused by previously unknown infectious agents with altered pathogenicity. Retroviruses are especially of concern since breeding susceptibility out of a species or barrier elimination of transmission is not possible at this time. Further, the risk of viral infection is known to be increased during transplantation by factors commonly associated with viral activation, e.g., immune suppression, graft-versus-host disease (GVHD), graft rejection, viral co-infection, and cytotoxic therapies. Since endogenous retroviruses are potential sources of infection, means of detecting their presence is essential. Primates and swine are potential candidate species for organ donation. Of the primates, chimpanzees and orangutans are endangered species. Baboons are too small to be an appropriate donor for humans. In addition, the relatively long gestation times and low reproduction rates of primates would hamper availability of organs for transplantation. Further, there is concern that xenografts from non-human primates would present considerable risk of transmission of pathogens and the consequent development of emerging infections, since several pathogens that cause disease are known to infect both humans and non-human primates.

The physiology of many organ systems of pigs has been shown to be highly similar to their human counterparts (Sachs, D. H. 1994. Veterinary Immunology & Immunopathology 43: 185-191). Swine have no reproductive season, are fecund, and have a relatively short gestation period. Through a selective breeding program over the past 20 years, partially inbred, miniature swine have been produced (Sachs et al. 1976. Transplantation 22: 559-567; Sachs, D. H. 1992. In Swine as Models in Biomedical Research, eds M. Swindle, D. Moody, and L. Phillips, pp. 3-15. Ames Iowa State Univ. Press; Sachs, 1994. Veterinary Immunology & Immunopathology 43: 185-191). These animals are similar in size to humans, each weighing about 100-150 kg at maturity. Further, herds of animals that are genetically well characterized and inbred at the major histocompatibility complex (MHC) are now available. Thus the species is considered a suitable source of such xenogeneic organs. Such use would obviate problems associated with the consideration of non-human primates as donors. However, the possibility of disease transmission from pigs to humans remains a concern, since pathogens such as retroviruses, http://www.ncbi.nim.nih.gov/ICTV/ are known to infect both species. Many microorganisms can be removed or eliminated by conventional barrier breeding methods. However, endogenous retroviruses that incorporate their DNA into the genetic material of the pig cannot be obliterated by these techniques.

Retroviruses constitute a large family of enveloped animal viruses with single stranded, positive sense RNA genomes (Weiss et al. 1984. In RNA Tumor Viruses, New York: Cold Spring Harbor Press; Levy, 1992-1995. In The Retroviridae, eds, F. C. Heinz and R. R. Wagner. New York: Plenum Press). Following infection of a target cell, the genomic RNA is converted to a double-stranded DNA form which becomes stably integrated into the chromosomal DNA of the host cell. Retroviruses have been classified into two categories depending on their mode of replication: exogenous, being horizontally transmitted from an animal to permissive cells of another animal by infectious routes, and endogenous, being inherited according to Mendelian expectations by subsequent generations as a normal part of the germlne DNA (reviewed by Coffin, 1982, “Endogenous Retroviruses”, in RNA Tumor Viruses, eds. R. Weiss, N. Teich, H. Varmus and J. Coffin. New York: Cold Spring Harbor Laboratory Press; Stoye and Coffin, 1985, “Endogenous Retroviruses”, in RNA Tumor Viruses, eds. R. Weiss, N. Teich, H. Varmus and J. Coffin. New York: Cold Spring Harbor Laboratory Press; Wilkinson et al. 1994, “Endogenous human retroviruses”, in The Retroviridae, J. Levy, ed., pp 465-535. New York: Plenum Press; Tchenio and Heidmann, 1991. J. Virol. 69: 1079-1084). The endogenous proviruses are subject to the same biological regulation as the rest of the chromosomal DNA that constitutes the genome and are present in the genomes of all cells of an organism. Despite the diversity of exogenous and endogenous retroviruses, they share a common structure, genome organization and many life-cycle features. Some strains of retroviruses are endogenous in one species and exogenous in others. Some retroviruses change their pathogenicity following interspecies transmission and may result in emerging infections, thus being parasitic in one host and symbiotic in another host.

Different abbreviations have been used for endogenous retroviruses (ERVs) (Lower et al. 1996. Proc. Natl. Acad. Sci. USA, 93: 5177-84). PERV, which is an abbreviation of porcine endogenous retrovirus, has been used for the endogenous retroviruses in the pig genome. PERV-A has been identified as a main safety concern for xenotransplantation.

Type C/Gamma retroviruses from cells of swine origin (PERV) have been characterized (Arida, E. and Hultin, T. 1977. Am. J. Public Health 67: 380; Armstrong et al. 1971. J. Gen. Virol. 10: 195-198; Benveniste, R. E. and Todaro, G. J. 1973. Proc. Natl. Acad. Sci. USA 70:3316-3320; Bouilant et al. 1975. J. Gen. Virol. 27: 173-180; Frazier, M. E. 1985. Arch. Virol. 83: 83-97; Lieber et al. 1975. Virology 66:616-619; Susuka et al. 1985. FEBS Lett. 183: 124-128; Susuka et al. 1986. FEBS Lett. 198: 339-343; Todaro et al. 1974. Virology 58: 65-74; Woods et al. 1973. J. Virol. 12: 1184-1186; Akiyoshi et al. 1998. J. Virol. 72: 4503-4507) but, as yet, no disease following infection by these viruses has been identified. A recent report demonstrated that PERV can infect human cells in vitro (Patience et al. 1997. Nature Medicine 3:276-282). A means of detecting the presence of retroviruses is essential.

Characterization of swine cells and cell lines has resulted in the identification of at least three subfamilies of PERV (PERV-A, -B, -C), (WO 97/40167; WO 97/21836; Le Tissier et al. 1997. Nature 389: 681-682; Czauderna et al. 1998: GenBank Accession Number Y17013). These sequences have distinct envelope (env) genes but share highly conserved sequences in the rest of the genome. Southern blot analysis of genomic DNA prepared from different pig tissues and cell lines (Patience et al. 1997. Nature Medicine 3:276-282) showed the presence of numerous loci in genomic DNA extracted from normal pig hearts and from pig cell lines. The Southern blot banding profile for DNA prepared from normal pig hearts is similar to that obtained from DNA of the pig cell lines and is typical of an endogenous inherited retrovirus suggesting heterogeneity with approximately 50 integration sites. These results were confirmed and extended to analysis of MHC-inbred miniature swine where the numbers of potentially full-length provirus copies are approximately 8 to 15 per genome for inbred and outbred swine and 10 to 20 in PK15 cells (Akiyoshi et al. 1998. J. Virol., 72:4503-4507).

The envelope gene determines the host range and cell tropism of PERV. The envelopes of PERV-A, -B, and -C are distinct. In particular, a high degree of amino acid differences in the VRA, VRB, and PRO regions in the SU glycoproteins is evident upon sequence comparison. Host range analyses using retrovirus vectors bearing corresponding envelope proteins showed that PERV-A and PERV-B envelopes have wider host range including several human cell lines as compared to PERV-C envelope, which has been shown to mediate entry into only two pig cell lines and, possibly, a single human cell line. All three strains of type C PERVs have been shown to infect pig cells. Receptors for PERV-A and PERV-B have been shown to be present on cells of some other species, including mink, rat, mouse and dog. Interference studies showed that the three PERV strains each use distinct receptors from each other and from those used by a number of other type C mammalian retroviruses (Takeuchi et al. 1998. J. Virol., 72: 9986-9991).

In accordance with the present invention, identification of the receptor used by this virus to infect human cells is a major step toward the understanding of the biology of this virus and for the safety of xenotransplantation. Receptor definition allows for the identification of infectable cells and therefore of possible sites of pathogenicity in vivo. In addition, receptor identification also facilitates the development of transgenic animal models of PERV infection which can be used to investigate the potential for horizontal transmission of PERV. Transmission between individuals is of particular importance for xenotransplantation as the potential spread of a novel zoonotic infection from a xenograft recipient to contacts and the general population represents a significant public health concern. If a human cell, especially a germline cell such as sperm or oocyte, displays a PERV receptor, then infection and possible incorporation of PERV DNA into human DNA may occur. In accordance with the present invention, having the DNA sequence allows expression of the protein and thus facilitates means, such as definition of small molecule inhibitors and generation of receptor inhibiting antibodies for administration, to block the receptor. Additionally, knowledge of the DNA sequence allows the production of model systems for testing anti-viral agents, antibodies, and small molecule drugs.

### BRIEF SUMMARY OF THE INVENTION

In one aspect, the present invention relates to polypeptide sequences for PERV receptors found on primate cells and polynucleotides encoding them.

In another aspect, the present invention relates to processes for using the PERV receptors of the invention as a means of generating blocking agents, such as antibodies or other molecules, for use in blocking attachment and/or infection of PERV to cells, especially human cells, as a means of preventing infection following xenotransplantation.

In a related embodiment, the present invention relates to a screening assay for identifying agents, such as chemical compounds, preferably antibodies or other molecules, that block, or otherwise interfere with, the binding of retroviruses, preferably PERVs, preferably PERV-A, to cells expressing PERV-receptors, especially where the blocking agent can be utilized in vivo.

In another embodiment, the present invention relates to recombinant cells that are infectable after being transfected with the gene for a receptor of the invention and subsequently expressing this receptor on the cell surface. Such cells are useful for in vitro screening assays according to the invention.

The present invention also relates to antibodies or other molecules, such as therapeutics, that bind to the receptors disclosed herein.

The present invention also relates to vectors, such as viruses and plasmids, and other nucleotide constructs that contain polynucleotides encoding the receptors disclosed herein and to cells, especially recombinant cells, engineered to express such receptors, especially where the cells do not express those receptors in the absence of such genetic engineering.

The present invention further relates to a small animal PERV infection model, such as a transgenic animal, utilizing the nucleotide sequences of the present invention to produce transgenic expression of PERV-A receptors. Such a model would find use in assessing the pathogenic consequences of PERV infection, in understanding the mode of transmission of PERV in vivo, and in the development of agents inhibiting such infection or transmission.

The present invention further relates to a process for blocking a PERV receptor, preferably PERV-A, on a cell, comprising contacting a cell expressing a PERV receptor with an agent that binds to the PERV receptor thereby blocking binding of PERV to the cell.

The present invention also relates to a process for protecting against PERV infection in a patient at risk of such infection comprising administering to the patient an effective amount of an agent that binds to PERV-A receptors thereby protecting against PERV infection.

### BRIEF DESCRIPTION OF THE DRAWINGS

FIG. 1 shows a schematic diagram of the envelope (ENV) region of PERV-A 14/220. SEQ ID NO: 1 and 3 are the ENV nucleotide sequences from different isolates from the ATG start codon through to the end of the TM region. The corresponding amino acid sequences of the envelope proteins are presented as SEQ ID NO: 2 and 4.

FIG. 2 shows the results of a northern blot demonstrating the expression of PERV-A receptor genes in a variety of tissues. The gels are spread over 3 blots with lanes numbered left to right as follows: A (upper blot) with lanes: 1 (brain), 2 (heart), 3 (skeletal muscle), 4 (colon), 5 (thymus), 6 (spleen), 7 (kidney), 8 (liver), 9 (small intestine), 10 (placenta), 11 (lung) and 12 (PBL); B (middle blot) with lanes: 13 (spleen), 14 (thymus), 15 (prostate), 16 (testes—dark), 17 (ovary), 18 (small intestine), 19 (colon), 20 (PBL); C (lower blot) with lanes: 21 (adrenal gland), 22 (bladder), 23 (marrow), 24 (brain), 25 (lymph node), 26 (mammary gland), 27 (prostate), 28 (spinal cord), 29 (stomach), 30 (thyroid), 31 (trachea) and 32 (uterus). The germlne cells (testis and ovary) highly expressed PERV receptors.

FIG. 3 graphically illustrates the reverse transcriptase (RT) activity found in the supernatant of SIRC cells (a cell line from rabbits) transfected with PHuR-A1 (SEQ ID NO: 11), PHuR-A2 (SEQ ID NO: 13), PBaR-A1 (SEQ ID NO: 15), or no receptor nucleotide sequence, after challenge with PERV-A 14/220. The results show that SIRC cells expressing one of PHuR-A1, PHuR-A2, or PBaR-A1 can support productive replication of PERV.

### DEFINITIONS

As used herein and except as noted otherwise, all terms are defined as given below.

In accordance with the present invention, the term “DNA segment” refers to a DNA polymer, in the form of a separate fragment or as a component of a larger DNA construct, which has been derived from DNA isolated at least once in substantially pure form, i.e., free of contaminating endogenous materials and in a quantity or concentration enabling identification, manipulation, and recovery of the segment and its component nucleotide sequences by standard biochemical methods, for example, using a cloning vector. Such segments are provided in the form of an open reading frame uninterrupted by internal nontranslated sequences, or introns, which are typically present in eukaryotic genes. Sequences of non-translated DNA may be present downstream from the open reading frame, where the same do not interfere with manipulation or expression of the coding regions.

“Isolated” in the context of the present invention with respect to polypeptides (or polynucleotides) means that the material is removed from its original environment (e.g., the natural environment if it is naturally occurring). For example, a naturally-occurring polynucleotide or polypeptide present in a living organism is not isolated, but the same polynucleotide or polypeptide, separated from some or all of the co-existing materials in the natural system, is isolated. Such polynucleotides could be part of a vector and/or such polynucleotides or polypeptides could be part of a composition, and still be isolated in that such vector or composition is not part of its natural environment. The polypeptides and polynucleotides of the present invention are preferably provided in an isolated form, and preferably are purified to homogeneity.

The polynucleotides and polypeptides disclosed in accordance with the present invention may be in “purified” form. The term “purified” does not require absolute purity; rather, it is intended as a relative definition, and can include preparations that are highly purified or preparations that are only partially purified, as those terms are understood by those of skill in the relevant art. For example, individual clones isolated from a cDNA library have been conventionally purified to electrophoretic homogeneity. Purification of starting material or natural material to at least one order of magnitude, preferably two or three orders, and more preferably four or five orders of magnitude is expressly contemplated. Furthermore, a claimed polypeptide which has a purity of preferably 0.001%, or at least 0.01% or 0.1%; and even desirably 1% by weight or greater is expressly contemplated.

The term “coding region” refers to that portion of a gene which either naturally or normally codes for the expression product of that gene in its natural genomic environment, i.e., the region coding in vivo for the native expression product of the gene. The coding region can be from a normal, mutated or altered gene, or can even be from a DNA sequence, or gene, wholly synthesized in the laboratory using methods well known to those of skill in the art of DNA synthesis.

In accordance with the present invention, the term “nucleotide sequence” refers to a heteropolymer of deoxyribonucleotides. Generally, DNA segments encoding the proteins provided by this invention are assembled from cDNA fragments and short oligonucleotide linkers, or from a series of oligonucleotides, to provide a synthetic gene which is capable of being expressed in a recombinant transcriptional unit comprising regulatory elements derived from a microbial or viral operon.

The term “expression product” means that polypeptide or protein that is the natural translation product of the gene and any nucleic acid sequence coding equivalents resulting from genetic code degeneracy and thus coding for the same amino acid(s).

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. When used in relation to polynucleotides, such terms refer to the products produced by treatment of said polynucleotides with any of the common endonucleases.

The term “fragment,” when referring to a coding sequence, means a portion of DNA comprising less than the complete coding region whose expression product retains essentially the same biological function or activity as the expression product of the complete coding region.

The term “primer” means a short nucleic acid sequence that is paired with one strand of DNA and provides a free 3′OH end at which a DNA polymerase starts synthesis of a deoxyribonucleotide chain.

The term “promoter” means a region of DNA involved in binding of RNA polymerase to initiate transcription.

The term “open reading frame (ORF)” means a series of triplets coding for amino acids without any termination codons and is a sequence (potentially) translatable into protein.

As used herein, reference to a DNA sequence includes both single stranded and double stranded DNA. Thus, the specific sequence, unless the context indicates otherwise, refers to the single strand DNA of such sequence, the duplex of such sequence with its complement (double stranded DNA) and the complement of such sequence.

In accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:

Percent Identity=100[1−(C/R)]

wherein C is the number of differences between the Reference Sequence and the Compared Sequence over the length of the alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid sequence in the Reference Sequence that is different from an aligned base or amino acid sequence in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

As known in the art “similarity” between two polypeptides is determined by comparing the amino acid sequence and its conserved amino acid substitutes of one polypeptide to the sequence of a second polypeptide.

The term “active fragment” when referring to a fragment of a polypeptide means a fragment that retains essentially the same biological function or activity as such polypeptide. Such a fragment is one that reacts, under suitable conditions, with PERV-A.

The term “immunogenic fragment” when referring to a fragment of a polypeptide means a fragment that reacts with an antibody specific for said polypeptide or that elicits production of such antibodies when administered to an immunocompetent animal, especially a human.

The term “analog” when referring to a polypeptide means a polypeptide that retains essentially the same biological function or activity of such polypeptide. As used herein an analog includes a proprotein which can be activated by cleavage of the proprotein portion to produce an active mature polypeptide.

The term “stringent conditions” means hybridization will occur only if there is at least 95% and preferably at least 97% identity between the sequences.

As used herein, the term “conservative amino acid substitution” is defined as an exchange within one of the following five groups.

I. Small aliphatic, nonpolar or slightly polar residues:

Ala, Ser, Thr, Pro, Gly;

II. Polar, negatively charged residues and their amides:

Asp, Asn, Glu, Gin;

III. Polar, positively charged residues:

His, Arg, Lys;

IV. Large, aliphatic, nonpolar residues:

Met, Leu, Ile, Val, Cys;

V. Large, aromatic residues:

Phe, Tyr, Trp.

### DETAILED DESCRIPTION OF THE INVENTION

In accordance with the present invention, novel primate cell surface receptors for PERV-A have been discovered. The nucleotides encoding these receptors include human sequences: SEQ ID NO: 11, mapping to human chromosome 8, SEQ ID NO: 13, mapping to human chromosome 17, and baboon sequence SEQ ID NO: 15. The human receptor of SEQ ID NO: 12 is encoded by the nucleotide sequence of SEQ ID NO: 11 (designated PHuR-A1 and wherein the open reading frame is residues 112-1449). The human receptor of SEQ ID NO: 14 is encoded by the nucleotide sequence of SEQ ID NO: 13 (wherein residues 79-1425 represent the open reading frame and the remaining sequences are the flanking sequences amplified by the primer sequences used to clone it). The baboon receptor of SEQ ID NO: 16 is encoded by nucleotide sequence SEQ ID NO: 15 (designated PBaR-A1 and representing the ORF). The sequence of SEQ ID NO: 9 (which encodes SEQ ID NO: 10) is the closest GenBank sequence wherein the open reading frame spans nucleotides 240-1577.

The receptor proteins disclosed herein according to the present invention contain 445 (SEQ ID NO: 12), 448 (SEQ ID NO: 14) and 448 (SEQ ID 16) amino acids and have similar transmembrane profiles to known receptor proteins. Identification of the PERV-A receptors has important implications for understanding of the safety of xenotransplantation and investigation of possible associated retroviral diseases, as well as prevention thereof. Percentage identities between the sequences of the invention and relative to known sequences are indicated in Table 1. FLJ11856 is a GenBank sequence (Accession No. MN024531). FLJ 10060 is a GenBank sequence with an open reading frame identical to that of human A2 (PHuR-A2, SEQ ID NO: 13) but having different flanking sequences. Percent identities were calculated using the sequences of the open reading frame portions of the sequences.

TABLE 1

| | | | | |
|---|---|---|---|---|
| PHuR-A1 | PHuR-A2 | PBaR-A1 | 11856 | 10060 |

NUCLEOTIDE PERCENTAGE IDENTITY

| PHuR-A1 | | 86 | 86 | 99 | 85 |
|---|---|---|---|---|---|
| PHuR-A2 | | | 96 | 86 | 99 |
| PBaR-A1 | | | | 86 | 96 |
| FLJ11856 | | | | | 86 |
| | PHuR-A1 | PHuR-A2 | PBaR-A1 | FLJ11856 | FLJ10060 |

AMINO ACID PERCENTAGE IDENTITY

| | | | | |
|---|---|---|---|---|
| PHuR-A1 | 86 | 85 | 99 | 86 |
| PHuR-A2 | | 95 | 86 | 99 |
| PBaR-A1 | | | 85 | 95 |
| FLJ11856 | | | | 86 |

In its broadest aspect, the present invention relates to viral receptors, especially PERV receptors, found on mammalian cells, especially primate cells, as well as to processes of using such polypeptides and the polynucleotides encoding them, including the full complements of these polynucleotides.

In one embodiment, the polynucleotides of the invention include the polynucleotide of SEQ ID NO: 11 as well as polynucleotides having nucleotide sequences that encode the amino acid sequence of SEQ ID NO: 12. Other embodiments thereof include sequences at least 85% identical to, preferably at least 90% identical to, most preferably at least 95% identical to, especially at least 98% identical to, and most especially having the sequence of SEQ ID NO:

In another embodiment, the polynucleotides of the invention include the polynucleotide of SEQ ID NO: 13 as well as polynucleotides having nucleotide sequences that encode the amino acid sequence of SEQ ID NO: 14. Other embodiments thereof include sequences at least 85% identical to, preferably at least 90% identical to, most preferably at least 95% identical to, especially at least 98% identical to, and most especially having the sequence of SEQ ID NO: 13.

In an additional embodiment, the polynucleotides of the invention include the polynucleotide of SEQ ID NO: 15 as well as polynucleotides having nucleotide sequences that encode the amino acid sequence of SEQ ID NO: 16. Other embodiments thereof include sequences at least 85% identical to, preferably at least 90% identical to, most preferably at least 97% identical to, especially at least 99% identical to, and most especially having the sequence of SEQ ID NO: 15.

The present invention also relates to an isolated polypeptide comprising a polypeptide having an amino acid sequence at least 95% identical to, preferably at least 96% identical, most preferably at least 98% identical to, and especially a polypeptide having the amino acid sequence of SEQ ID NO: 12, 14, 16 or 17. In a most preferred embodiment, where the sequences of the polypeptides of the invention differ from those of SEQ ID NO: 12, 14, 16 or 17, such differences are due solely to conservative amino acid substitution(s).

Fragments or portions of the polypeptides of the present invention may be employed for producing the corresponding full-length polypeptide by peptide synthesis; therefore, the fragments may be employed as intermediates for producing the full-length polypeptides. Fragments or portions of the polynucleotides of the present invention may be used to synthesize full-length polynucleotides of the present invention.

In specific embodiments, the polynucleotides of the invention may be coding sequences or anti-coding sequences, which are necessarily complementary to each other. The polynucleotides of the invention may be DNA, such as a cDNA, or RNA. Fragments of the polynucleotide sequences disclosed herein may be used as hybridization probes for a cDNA or DNA library to isolate the full-length gene and to isolate other genes which have a high sequence similarity to the gene or similar biological activity. Probes of this type have at least 15 contiguous bases, preferably at least 30 bases and may contain, for example, 50, 100 or more bases. The probes may also be the polynucleotides comprising the nucleotide sequences of SEQ ID NO: 9, 11, 13, or 15. The probe may also be used to identify a cDNA clone or DNA clone corresponding to a full length transcript and a genomic clone or clones that contain the complete gene including regulatory and regions, exons, and introns. An example of a screen comprises isolating the coding region of the gene by using the known DNA sequence to synthesize an oligonucleotide probe. Labeled oligonucleotides having a sequence complementary to that of the receptor gene of the present invention are used to screen a library of mammalian, especially human, DNA, genomic DNA or mRNA to determine which members of the library the probe hybridizes to.

The present invention further relates to polynucleotides which hybridize to the hereinabove-described polynucleotide sequences if there is at least 80% sequence identity, preferably at least 90% identity, more preferably at least 95% identity, and most preferably at least 98% identity between the sequences, or the complements of these. The present invention particularly relates to polynucleotides which hybridize under stringent conditions to the hereinabove-described polynucleotides. The polynucleotides which hybridize to the hereinabove described polynucleotides in a preferred embodiment encode polypeptides which retain substantially the same biological function or activity as the mature polypeptide comprising the amino acid sequence of SEQ ID NO: 12, 14 or 16.

The present invention further relates to a process for detecting the presence of a PERV-receptor gene in a cellular genome comprising contacting a sample of said genome with a probe comprising at least 15 contiguous nucleotides of a novel polynucleotide disclosed herein, preferably at least 30 contiguous nucleotides, more preferably at least 50 contiguous nucleotides, most preferably at least 80 contiguous nucleotides and especially at least 100 contiguous nucleotides, wherein said contiguous nucleotides comprise a sequence characteristic of the gene for the PERV receptor. In a highly preferred embodiment, said probe comprises the entire sequence of SEQ ID NO: 9, 11, 13, or 15.

The present invention also relates to polypeptides that can act as PERV receptors, especially PERV-A, most especially human and baboon receptors.

A polypeptide of the present invention may be a recombinant polypeptide, a natural polypeptide or a synthetic polypeptide, preferably a recombinant polypeptide.

The fragment, derivative or analog of the polypeptide may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as a leader or secretory sequence or a sequence which is employed for purification of the mature polypeptide or a proprotein sequence. Such fragments, derivatives and analogs are deemed to be within the scope of those skilled in the art from the teachings herein.

The polypeptides and polynucleotides of the present invention are preferably provided in an isolated form, and preferably are purified to homogeneity.

The polypeptides useful in practicing the invention also include active fragments and/or immunogenic fragments of these polypeptide receptors provided that said fragments are capable of specifically binding to endogenous retroviruses, especially PERVs, most especially PERV-A. For example, fragments of the polypeptides of SEQ ID NO: 10, 12, 14, 16 or 17 may be employed for this purpose.

The polypeptides useful in the present invention include polypeptides having the amino acid sequence of SEQ ID NO: 12, 14, or 16 as well as polypeptides differing from this sequence only by one or more conservative amino acid substitutions.

In another aspect, the present invention relates to processes for using the PERV receptors of the invention in models for the infectivity by retroviruses, preferably PERVs, most preferably PERV-A, in both in vitro and in vivo measurements of viral infectious capability (i.e., as a basis for screening assays to identify compounds such as antibodies and small molecules that interfere with PERV binding.

In one embodiment, the present invention relates to a screening assay for identifying agents, including chemical compounds and other molecules, such as antibodies and small molecules, that are able to block, or otherwise interfere with, the binding of retroviruses, preferably PERVs, preferably PERV-A, to cells expressing the PERV-receptors of the invention, especially where that blocking can be utilized in vivo.

In another embodiment, the present invention relates to recombinant cells not naturally susceptible to infection by PERVs, preferably PERV-A, but which are infectable after being transfected with polynucleotides encoding the receptors of the invention and subsequently expressing this receptors on the cell surface. Such cells are useful for in vitro screening assays for other viruses capable of infecting human cells as well as for agents, especially antibodies and small molecules, capable of blocking or otherwise interfering with such binding.

In yet another aspect, the present invention relates to vectors and vector constructs (for example polynucleotide sequences comprising a promoter and a coding sequence) capable of expression of an amino acid sequence of the present invention.

In accordance with the foregoing, the present invention relates to a process for identifying a compound that prevents, inhibits, interferes with, competes with, or otherwise reduces the rate or extent of, porcine endogenous retrovirus (PERV)-binding to a cell susceptible to such binding, comprising:

(a) contacting a PERV-A receptor with a compound, such as an antibody or small molecule, under conditions promoting binding of the compound to the PERV-A receptor, and

(b) detecting binding of the compound to the PERV-A receptor,

thereby identifying the compound as one that prevents, inhibits, interferes with, competes with, or otherwise reduces the rate or extent of PERV-binding to the cell.

In a preferred embodiment thereof, the PERV-A receptor comprises a polypeptide having an amino acid sequence selected from the group consisting of SEQ ID NO: 10, 12, 14, and 16 or an active fragment of such polypeptide (i.e., a fragment able to react with, preferably to selectively or specifically bind, PERV, especially PERV-A).

The present invention specifically contemplates a process wherein the PERV receptor is part of a membrane or some type of lipid bilayer, including one from a natural source such as a cell membrane or one from a wholly synthetic source, such as a liposome. In a preferred embodiment, such lipid bilayer or membrane is part of an intact cell, most preferably a human cell. Such cell may be of natural origin or may be a recombinant cell that has been engineered to express a PERV receptor polypeptide on its surface, preferably where the cell does not express the polypeptide prior to being engineered. Such engineering may include structural engineering, whereby a preformed receptor has been inserted into a cell, or genetic engineering, whereby a cell has been transfected with a polynucleotide, or a vector comprising a polynucleotide, that encodes a PERV receptor, preferably the PERV-A receptors of the invention, so that the resulting transfected cell expresses the receptor polypeptide.

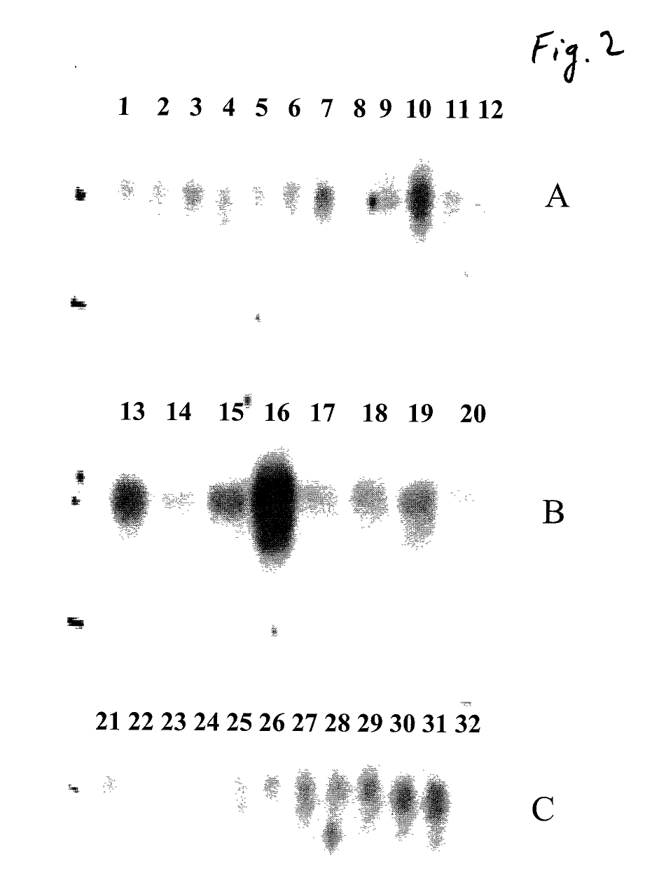

The invention also contemplates the insertion of the polynucleotide into the genome of a cell to effect expression of the PERV receptor. In addition, embodiments of the invention include situations where a cell already expresses a PERV receptor, preferably a PERV A receptor, but is engineered, so that the resulting transfected or recombinant cell now expresses such PERV receptors at a higher level than without such engineering. Cells useful in the processes of the invention can include cells derived from any of the tissues disclosed in FIG. 2 that express PERV receptors, such as testis and ovary.

Such recombinant cells may result from transfection with any of the polynucleotides disclosed herein that encode PERV receptors, especially those receptors of SEQ ID NO: 10, 12, 14 and 16, or which encode the consensus sequence of SEQ ID NO: 17, the latter resulting from alignment of the two human (SEQ ID NO: 12 and 14) and baboon (SEQ ID NO: 16) amino acid sequences.

The present invention further relates to a process of identifying compounds inhibiting PERV binding to cells, as already described, but wherein the contacting occurs in the presence of PERV and under suitable conditions (i.e., conditions promoting binding of said PERV to the cell or membrane or other lipid bilayer) and detecting a decrease, or interference with, or even complete blockage, in binding of PERV to the cell as compared to when the compound is not present thereby identifying a compound that interferes with PERV-binding.

In one such embodiment, the invention may be used in a process for screening a plurality of chemical compounds for ability to prevent, inhibit, interfere with, compete with, or otherwise reduce the rate or extent of PERV-binding comprising:

(a) contacting a source of PERV receptors, such as a membrane or a cell expressing on its surface a polypeptide, such as the PERV-receptors disclosed herein, with the compound under conditions suitable for, or capable of, promoting binding of the compound to the PERV receptor, and

(b) detecting specific binding of the chemical compound to the PERV receptor,

thereby identifying a compound capable of interfering with, or of blocking (i.e., completely preventing) PERV-binding.

In a preferred embodiment of such process, the PERV receptor is one comprising all or an active portion or fragment of the amino acid sequence of SEQ ID NO: 10, 12, 14, or 16.

In another preferred embodiment, the present invention relates to a process for identifying an agent that interferes with porcine endogenous retrovirus (PERV)-binding to a PERV-receptor comprising:

(a) contacting in vitro a polypeptide of SEQ ID NO: 10, 12, 14, 16 or 17 with the compound under conditions that promote binding of the compound to said polypeptide, and

(b) detecting specific binding of the compound to the polypeptide of step (a),

thereby identifying a compound that prevents PERV-binding to the PERV receptor.

In other embodiments of the processes of the invention, the PERV receptor may include polypeptides comprising amino acid sequences at least 85%, preferably at least 90%, most preferably at least 95%, especially at least 98% identical, or even identical to SEQ ID NO: 14. In preferred embodiments, this would include any of the sequences of SEQ ID NO: 10, 12, 14, 16, or 17.

In another aspect, the present invention relates to a process for blocking a PERV receptor on a cell comprising contacting a cell expressing a PERV receptor with an agent that binds to the PERV receptor thereby blocking binding of PERV to the cell. In such process, the PERV receptor is preferably a PERV-A receptor. Other preferred embodiments include processes wherein the cell is a human cell and/or the agent is an antibody, preferably an antibody that reacts with, and most preferably is specific for, a polypeptide comprising the amino acid sequence of SEQ ID NO: 10, 12, 14, 16, or 17 or wherein such antibody is an antibody that reacts with an immunogenic fragment of a polypeptide comprising the amino acid sequence of SEQ ID NO: 10, 12, 14, 16 or 17.

In a related embodiment, the present invention also contemplates a process for protecting against PERV infection in a patient at risk of such infection comprising administering to the patient an effective amount of an agent that binds to PERV-A receptors thereby protecting against PERV infection. In a preferred embodiment thereof, the PERV is PERV-A.

In highly useful embodiments of the processes of the invention, the source of the PERV infection is a tissue used for xenotransplantation. Thus, where an organ or tissue is to be transplanted from an animal donor, especially miniature swine, the invention is useful in determining the likelihood of infection of the human recipient as well as providing a means of blocking the receptors on cells of the recipient so that any virus that may be undetected in the donor tissue will be unable to infect the recipient following blockage of the receptors on the cells of the recipient. Highly advantageous for such purposes is an antibody, such as a monoclonal or recombinant antibody, that reacts with, preferably is specific for, a PERV receptor, especially PERV-A, and most especially a PERV-A receptor as disclosed herein.

In a preferred embodiment, such an antibody reacts with a PERV-A receptor that comprises the amino acid sequence of SEQ ID NO: 10, 12, 14, 16, or 17 or with an active fragment of such polypeptide wherein said immunogenic fragment is characteristic of PERV-A. It is of course to be anticipated that an antibody that reacts with a PERV-A receptor as disclosed herein will also react with, even be specific or selective for, polypeptides and immunogenic or active fragments thereof that may differ somewhat in amino acid sequence from the sequences for receptors as disclosed herein but will nevertheless succeed in blocking binding of PERV to cells, such as the cells of a xenotransplant recipient, and thereby protect against infection of the patient by PERV, especially PERV-A. Thus, antibodies useful in practicing the invention include any antibodies that react with, especially antibodies that are selective or specific for, polypeptides comprising the amino acid sequence of SEQ ID NO: 10, 12, 14, 16 or 17.

The agents useful in the present invention for blocking PERV binding and/or protecting against, or preventing, PERV infection may conveniently be present in the form of a composition. The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids such as water, saline, glycerol and ethanol, and the like, including carriers useful in forming sprays. A thorough discussion of pharmaceutically acceptable carriers, diluents, and other excipients is presented in REMINGTON'S PHARMACEUTICAL SCIENCES (Mack Pub. Co., N.J. current edition).

The present invention also relates to antibodies that react with the PERV receptors disclosed herein. Such antibodies react with PERV receptors, especially the PERV-A receptors disclosed herein. These antibodies may be monoclonal antibodies or antibodies specifically designed for their reaction with such PERV receptors. The antibodies may be selective for the PERV receptors disclosed herein or highly specific for such receptor polypeptides. Such antibodies find use in the assays of the invention, in the detection of PERV receptors on tissues, or in the protection against PERV infection by binding to, and tying up of, available PERV receptors in tissues of an animal, especially a human patient, at risk of PERV infection or otherwise prior to exposure to a potential source of PERV infection, such as prior to, during or after transplantation of an organ, including tissues or cells thereof. In a preferred embodiment, such transplantation is xenotransplantation.

In specific embodiments, such antibodies may react with polypeptides comprising amino acid sequences at least 87%, preferably at least 90%, most preferably at least 95%, especially at least 98%, identical to SEQ ID NO: 14. In highly preferred embodiments, such antibodies react with polypeptides comprising the amino acid sequences of SEQ ID NO: 10, 12, 14, 16 or 17. These antibodies may also react with active fragments of any of these polypeptides, where the term “active fragment” means a portion, fragment or segment of the polypeptide that reacts with the antibody, such as where the antibody is selective or specific for such fragments or where such fragments, if administered to an animal different from the source of the polypeptide will elicit the production of antibodies that react with the polypeptide or active fragment. Thus, such fragments include immunogenic fragments.

In specific embodiments, such an antibody reacts with any of the polypeptides disclosed herein and which polypeptides are capable of binding endogenous retroviruses, especially PERV, most especially PERV-A. Such antibodies include humanized and/or recombinant antibodies.

In other specific embodiments of inhibiting agents, such a small molecule reacts with any of the polypeptides disclosed herein, which polypeptides are capable of binding endogenous retroviruses, especially PERV, most especially PERV-A. Such small molecules include those found utilizing a model system formed by transgenic engineering of cells and of small animal models incorporating a nucleotide sequence of the present invention, in particular SEQ ID NO: 11, 13, and 15.

With the advent of methods of molecular biology and recombinant technology, it is now possible to produce antibody molecules by recombinant means and thereby generate gene sequences that code for specific amino acid sequences found in the polypeptide structure of the antibodies. Such antibodies can be produced by either cloning the gene sequences encoding the polypeptide chains of said antibodies or by direct synthesis of said polypeptide chains, with in vitro assembly of the synthesized chains to form active tetrameric (H₂L₂) structures with affinity for specific epitopes and antigenic determinants. This has permitted the ready production of antibodies having sequences characteristic of neutralizing antibodies from different species and sources.

Regardless of the source of the antibodies, or how they are recombinantly constructed, or how they are synthesized, in vitro or in vivo, using transgenic animals, such as cows, goats and sheep, using large cell cultures of laboratory or commercial size, in bioreactors or by direct chemical synthesis employing no living organisms at any stage of the process, all antibodies have a similar overall 3 dimensional structure. This structure is often given as H₂L₂ and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity.

The variable regions of either H or L chains contains the amino acid sequences capable of specifically binding to antigenic targets. Within these sequences are smaller sequences dubbed “hypervariable” because of their 20 extreme variability between antibodies of differing specificity. Such hypervariable regions are also referred to as “complementarity determining regions” or “CDR” regions. These CDR regions account for the basic specificity of the antibody for a particular antigenic determinant structure.

The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The accepted CDR regions have been described by Kabat et al, J. Biol. Chem. 252:6609-6616 (1977).

In all mammalian species, antibody polypeptides contain constant (i.e., highly conserved) and variable regions, and, within the latter, there are the CDRs and the so-called “framework regions” made up of amino acid sequences within the variable region of the heavy or light chain but outside the CDRs.

The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain. Such antibodies may also include fragments, such as Fab and F(ab₂)′ fragments, capable of reacting with and binding to any of the polypeptides disclosed herein as being receptors. Such antibodies may be able to block activation or alter activation of said receptors.

Thus, the present invention relates to a process for determining the presence of a PERV-binding site on a cell comprising contacting a cell with an antibody specific for a polypeptide comprising an amino acid sequence of SEQ ID NO: 10, 12, 14, 16 or 17 and detecting specific binding of said antibody to said cell wherein said binding indicates the presence on said cell of a PERV binding site. In a preferred embodiment, said PERV is PERV-A and said cell is a human cell.

The present invention also relates to vectors, such as viruses and plasmids, that contain polynucleotides encoding the receptors disclosed herein and which, after insertion into a cell, such as a human cells, confer upon that cell the ability to express the receptor polypeptide of the invention, thereby becoming susceptible to PERV infection.

Thus, in one embodiment, the present invention relates to a vector or vector construct comprising any of the polynucleotides disclosed herein, preferably a polynucleotide with a nucleotide sequence selected from SEQ ID NO: 11, 13, and 15. In preferred embodiments, such vector may be a virus or a plasmid, preferably a plasmid. The present invention also contemplates cells comprising such vectors and vector constructs, such as where such cells are bacterial or mammalian cells, preferably mammalian cells, most preferably human cells. Such cells may also be recombinant cells, such as where the cells do not normally express the receptors disclosed according to the invention but have been transfected with a polynucleotide of the invention so as to express either of said receptors on their surface. Such recombinant cells are specifically contemplated by the invention apart from their use in the processes disclosed herein.

In a preferred embodiment, the present invention relates to a mammalian cell, especially a recombinant cell, expressing on its surface a receptor comprising the polypeptide of SEQ ID NO: 12, 14, 16 or 17.

The present invention further provides processes for the more precise determination of the likelihood of transmission of PERV beyond the xenograft recipient. In addition, identification of homologues present in other animal species and their associated expression profile facilitates more specific determination of PERV infectivity in in vivo animal models.

Thus, the present invention also relates to a transgenic animal comprising cells into whose genome has been inserted a polynucleotide encoding a PERV-A receptor and which cells express said receptor on their surface but wherein said animal does not express said receptor absent said insertion. In a preferred embodiment thereof, the transgenic animal has a genome comprising a polynucleotide that encodes a polypeptide of claim 8, 9 or 10, most preferably wherein said polypeptide comprises the amino acid sequence of SEQ ID NO: 16. In especially preferred embodiments, said polynucleotide comprises the nucleotide sequence of SEQ ID NO: 11, 13 or 15. Said transgenic animal may advantageously be any small mammal, such as a mouse, a rat, a hamster or other small rodent, especially wherein said animal does not normally express a PERV receptor, such as a PERV-A receptor. In some such embodiments, where the transgenic animal otherwise expresses PERV receptors, such as PERV-A, the animal may have been engineered to over-express such receptors on one or more of its cells, or cell types. Such expression may be restricted to one or more cells or cell types, or one or more tissues or tissue types, or one or more organs of the transgenic animal.

In accordance therewith, the present invention further relates to a process for identifying a compound that protects against PERV infection comprising administering to a transgenic animal as disclosed herein a compound identified as interfering with PERV binding using any of the screening processes of the invention, then challenging said animal with a source of PERV and then determining that said animal does not exhibit the symptoms of PERV infection compared to when said compound has not been administered thereby identifying a compound that protects against PERV infection.

The present invention further relates to a process for determining the presence of a PERV-binding site on a cell comprising contacting a cell with an anti-PERV-receptor antibody and detecting specific binding of said antibody to said cell wherein said binding indicates the presence on said cell of a PERV binding site. In a preferred embodiment, said PERV is PERV-A. Antibodies useful in practicing the invention are those specific for the PERV-receptors disclosed herein, especially antibodies specific for polypeptides having the amino acid sequence of SEQ ID NO: 10, 12, 14, 16 or 17 including immunogenic fragments thereof (meaning fragments that elicit the production of antibodies against PERV, especially PERV-A, when administered to an immunologically competent animal). In a preferred embodiment, the PER-A receptor is a human or a baboon PERV-A receptor. It should be noted that the same PERV can infect both human and baboon cells.

The present invention also relates to a process that comprises a method for producing a product comprising identifying an agent according to one of the disclosed processes for identifying such an agent (i.e., the therapeutic agents identified according to the assay procedures disclosed herein) wherein said product is the data collected with respect to said agent as a result of said identification process, or assay, and wherein said data is sufficient to convey the chemical character and/or structure and/or properties of said agent. For example, the present invention specifically contemplates a situation whereby a user of an assay of the invention may use the assay to screen for compounds having the desired enzyme modulating activity and, having identified the compound, then conveys that information (i.e., information as to structure, dosage, etc) to another user who then utilizes the information to reproduce the agent and administer it for therapeutic or research purposes according to the invention. For example, the user of the assay (user 1) may screen a number of test compounds without knowing the structure or identity of the compounds (such as where a number of code numbers are used the first user is simply given samples labeled with said code numbers) and, after performing the screening process, using one or more assay processes of the present invention, then imparts to a second user (user 2), verbally or in writing or some equivalent fashion, sufficient information to identify the compounds having a particular modulating activity (for example, the code number with the corresponding results). This transmission of information from user 1 to user 2 is specifically contemplated by the present invention.

The present invention further relates to a process for protecting against PERV-infection in an animal comprising administering to an animal at risk of said infection, an effective amount of a compound (i.e. chemical or antibody) first identified as having such protective activity using one of the assay procedures disclosed herein for screening such compounds for such protective activity. In a preferred embodiment, the PERV to be protected against is PERV-A.

The present invention also relates to a process for treating an animal for PERV infection comprising administering to an animal afflicted therewith a therapeutically effective amount of a compound first identified as having such protective activity using one or more of the assay procedures disclosed according to the present invention. In a preferred embodiment, the PERV infection to be treated is PERV-A.

The present invention will now be further described by way of the following non-limiting example but it should be kept clearly in mind that other and different embodiments of the methods disclosed according to the present invention will no doubt suggest themselves to those of skill in the relevant art.

### EXAMPLE 1

### Cloning and Sequence Analysis of PERV-A Receptor

The cloning procedure consists of three critical elements, a high titer selectable PERV-A, a high titer retroviral cDNA library, and a cell line essentially uninfectable by the high titer PERV-A.

A human cDNA library derived from HeLa cells was purchased (Clontech pantropic retroviral expression system, Palo Alto, Calif.) and a high titer virus stock of this library was produced carrying the VSV envelope proteins according to the manufacturer's instructions. Briefly, approximately 10 μg of the HeLA cDNA library DNA was co-transfected into the VSV packaging cells (2×10⁶ cells in a 100-mm tissue culture plate) using Lipofectamine (Gibco BRL). Culture supernatant was harvested from the cells at 24 hour intervals up to 5 days, 0.45 um filtered and stored at −80° C. until use.

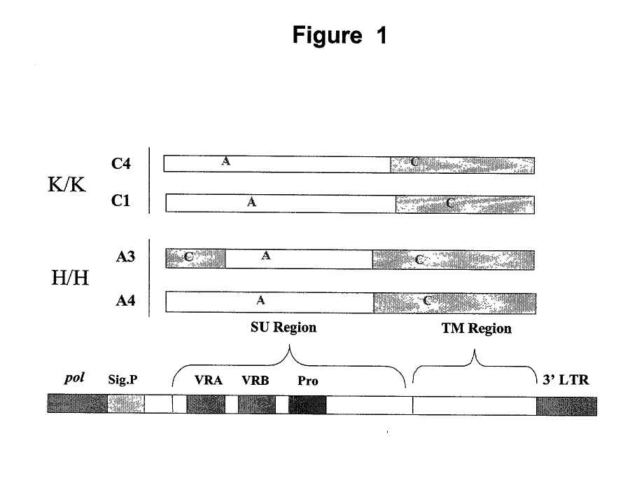

PERV-A isolates were identified as a result of transmission assays performed using PERV released from miniature swine PBMC (peripheral blood mononuclear cells), that grew to high titer in human 293 cells. This replication competent PERV (derived from pig 12005) represents a recombinant between PERV-A and PERV-C. In-house, this virus is termed PERV-A 14/220. Critical for the tropism of this virus stock is that the virus possesses VRA, a region derived from PERV-A i.e. the region that determines cell tropism. This VRA region of PERV-A (see FIG. 1) is recombined with the Tm envelope region, as well as the gag and pol genes of PERV-C. The ENV gene of sequenced isolates are represented by SEQ ID NO: 1 and 3.

In order to confer a selectable phenotype on the PERV-A 14/220 stock, the infected 293 cells were transfected with the retroviral vector pLN (A. D. Miller, Fred Hutchinson Research Institute) which carries a G418 resistance gene, using Lipofectamine (Gibco BRL) according to the manufacturer's instructions. Cells successfully transduced were selected by challenging the PERV infected 293 cells with G418. In addition to this PERV-A 14/220/Neo, PERV-A 14/220/LacZ was produced (also using the 293 cells infected by the 14/220-293PERV) by transduction of the infected 293 cells using standard methodology with the supernatant of TELCeBGALV cells. These cells contain the β-galactosidase gene in the retroviral vector MFGnIsLacZ. Due to the sequence similarity between the MLV-based retroviral vectors and PERV, the vectors efficiently compete with PERV transcripts for packaging into PERV particles and therefore result in the formation of PERV viruses that deliver either the LacZ or Neo markers.

Infection interference assays were used to determine the receptors used by the viruses present in the PERV-A 14/220 culture. Table 2 shows data confirming that the recombinant PERV uses the same receptor molecules as non-recombinant molecular clones of PERV-A. To determine whether cell lines were infectable by PERV-A 14/220, sub-confluent candidate cell lines were challenged with undiluted PERV-A 14/220 or PERV-A 14/220/Neo (approx. 10⁵ TCID₅₀/ml).

Brief Methodology

Human 293 cells infected by molecular clones of PERV-A, PERV-B, or PERV-A 14/220 were challenged with the 0.45 μm filtered culture supernatant of 293 PERV-A 14/220 as well as control viruses carrying the retroviral vector (MFGnIsLacZ) that encodes for β-galactosidase activity. If the LacZ pseudotype virus uses the same receptor as the virus already present in the infected target cell, then the titer of the LacZ pseudotype virus will be drastically reduced.

TABLE 2

| | Approximate LacZ pseudotype titer IU/ml | | | | |
|---|---|---|---|---|---|
| | TelCeb- | | | | |
| Target | PERV- | PERV-B/ | PERV-A/ | BTI 13/ | PERV-A |
| Cell Line | A18 | LacZ | LacZ | LacZ | 14/220/LacZ |
| 293 | 400 | 200 | 4 | 10³ | 10³ |
| 293 + PERV-B | 200 | <4 | 8 | 10³ | 10³ |
| 293 + PERV-A | <4 | 200 | <4 | 12 | 20 |
| 293 + 14/ | <4 | 200 | <4 | <4 | <4 |
| 220293PERV | | | | | |

To identify cell lines essentially uninfectable by PERV-A, multiple cell lines were challenged with undiluted PERV-A 14/2201Neo virus in the presence of 8 ug/ml polybrene using standard coculture techniques. The targeted cells were cultured in the presence of G418 at a concentration that kills wild-type cells. If colonies of cell grew in the presence of G418 this was therefore due to infection by the PERV-A 14/220/Neo virus and these cells were disregarded for further analysis. Of the cells screened, only SIRC and NIH3T3 appeared uninfectable by this methodology. Due to practical considerations, SIRC cells were used as the line of choice for the receptor cloning procedure. Growth medium for all SIRC cells was Minimal Essential Medium supplemented with 10% fetal bovine serum and antibiotics. All other cell lines were grown in Dulbecco's Modified Eagles Medium with the same supplements.

SIRC cells were plated in 100 mm dishes and cultures at 50% confluency. Undiluted VSV library supernatant was added to the cells for approximately 12 hours to allow the particles to infect the SIRC cells and introduce random cDNAs. The cells were then left in culture for 48 hours to allow expression of the cDNAs. The SIRC cells were then challenged with undiluted supernatant of 293 PERV-A 14/220/Neo cells and 8 μg/ml polybrene for 12 hours and then left in culture for a further 48 hours in order for the infectable cells to express the Neo resistance gene. The cells were then exposed to 1200 μg/ml G418 that was refreshed every 48 hours until the control cells (wild-type SIRC cells that had been exposed to the PERV supernatant but had not received the cDNA preparation) were dead, and colonies had grown out of the PERV-challenged cells. These resistant clones were re-challenged with undiluted supernatant from the 293 PERV-A 14/220/LacZ cells. Of 12 colonies, one was reproducibly infectable as determined by standard LacZ staining procedures (Cosset et al., 1995 J. Virol, 69: 7430-6) DNA was purified from these cells and PCR was performed under the following conditions using the following primers and conditions, to include 30 cycles of: 95° C. 10 secs, 55° C. 45 secs, 72° C. 3 mins:

| | | |
|---|---|---|
| PLib5 | 5′ agccctcactccttctctag 3′ | (SEQ ID NO: 5) |
| LIBMCSR1 | 5′ gatgtttggccgaggcgg 3′ | (SEQ ID NO: 6) |

From this PCR a product was ligated into the pTOPO vector (Invitrogen, Carlsbad, Calif.) using the manufacturer's instructions. This construct was digested with Not1-Not1 restriction enzyme and sub-cloned into Not1 digested pCDNA3 vector using standard ligation and bacterial transformation technologies. DNA of this construct was prepared and transfected into SIRC and NIH3T3 cells (ATCC; i.e. cells uninfectable by PERV-A) using Lipofectamine according to the manufacture's instructions. Transfected cells were selected using 1200 ug/ml G418 and, once cultures had expanded, were challenged with PERV-A 14/220/LacZ. These challenged transfected cells were found to be infectable by using LacZ staining. Therefore, this molecule confers infectability by PERV and is a receptor for PERV-A.

Sequence analysis of the PERV-A receptor has revealed the nucleotide (SEQ ID NO: 11) and protein (SEQ ID NO: 12) sequences which demonstrate near identity to a hypothetical protein (FLJ 1856) identified in human melanoma cells (SEQ ID 9, 10) (GenBank Accession No. NM_{—}024531). This gene sequence encoding FLJ11856 has been mapped to chromosome 8 by the human genome project. The mRNA record is supported by experimental evidence; however, the coding sequence is predicted. The reference sequence was derived from BC002917.1.

The sequence of SEQ ID NO: 12 is the sequence of a PERV-A receptor according to the invention. It is encoded by the nucleotide sequence of SEQ ID NO: 11 with a reading frame having start codon “ATG” at residues 112-114.

Comparison of the above nucleotide sequence to the human genome database identified a molecule which showed significant sequence similarity to GenBank Accession No. XM_{—}008527 which is defined as Homo sapiens hypothetical protein FLJ10060 mRNA and which maps to chromosome 17.

A comparison of the hydrophobicity plots of the proteins disclosed herein indicates that the molecules are extremely similar. The proteins are likely to be present in the plasma membrane of a cell and therefore possibly be involved with PERV entry. The ability of the FLJ10060 molecule to mediate PERV infection was also determined. Briefly, the molecule was cloned following PCR from a oligo dT cDNA preparation from 293 cells (produced by known methods) using PCR with the primers CCAAAGCATCTTTGGACCTACC (SEQ ID NO: 7) and TCACGATGAAGACAGGTGGG (SEQ ID NO: 8). The product (amino acid SEQ ID NO: 14 and nucleotide SEQ ID NO: 13) was (1) cloned into pTopo (2) cloned into pCDNA3, and (3) transfected into SIRC cells, following the same methodology as described for FLJ 1856. These results indicate that 293 cells express the putative FLJ10060 receptor molecule. Because this receptor was shown to mediate PERV-A infection (see Tables 2 and 3) the isolation of the FLJ10060 cDNA from 293 cells can be taken as proof that 293 cells express a molecule that represents a PERV-A receptor.

Alignment of the nucleotide sequences of FLJ10060 and FLJ11856 identified areas of sequence conservation which we used to isolate a novel homolog (termed PBaR-A2) from baboon testes RNA. PBaR-A2 was cloned using hemi-nested PCR from a oligo-dT primed cDNA prepared from baboon testes RNA:Baboon PCR 1^{st} round 5′-GTKACCTTYGCYYKWCCTGG-3′ (SEQ ID 20), 5′-GGAGYKGGGTCCCCACCTG-3′ (SEQ ID 21): 2^{nd} round 5′-AATGGCAGCACCYMCGC-3′ (SEQ ID 22), 5′-TCAGGGGCCACAGGGGTC-3′ (SEQ ID 23); 95° C. 10 s, 55° C. 30 s, 72° C. 120 s. The product was (1) cloned into pTopo (2) cloned into pCDNA3, and (3) transfected into SIRC cells, following the same methodology as described for FLJ 1856 and FLJ10060.

SIRC cells expressing FLJ11856, FLJ10060, and PBaR-A2 were infected with 14/220-293PERV/LacZ as described previously (Cosset et al., 1995 J. Virol, 69: 7430-6) and yielded results indicating that the molecules can behave as a receptor for PERV-A. Results are shown in Table 3.

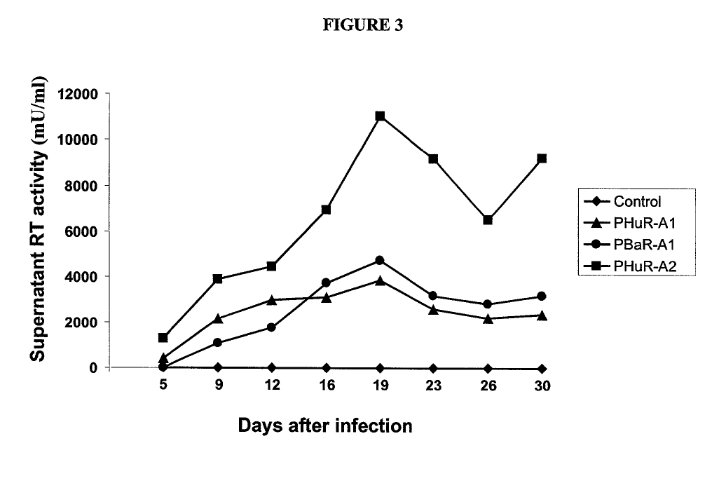

In addition the presence of any of these receptor molecules resulted in the ability of the SIRC cells to support PERV replication, a property not associated with wild-type SIRC cells. Briefly, cells were exposed to 0.45 μm filtered culture supernatants of replication competent 293 PERV-A 14/220 for 6 hours in the presence of 8 μg/ml polybrene. The cultures were maintained for three weeks and the reverse transcriptase (RT) activity present in the culture supernatants was measured. The presence of RT is an indication of virus replication. Results are shown in FIG. 3.

TABLE 3

| Target | Approximate LacZ pseudotype titer IU/ml | | |
|---|---|---|---|
| Cell Line | PERV-A 14/220 | TelCeB PERV-A18 | BTI 13* |
| IOWA | 8 × 10⁴ | 1 × 10⁴ | 6.4 × 10³ |
| 293 | 8 × 10⁴ | 40 | 7 × 10³ |
| HT1080 | 4 × 10⁴ | <4 | 8.4 × 10³ |
| SIRC | <4 | <4 | <4 |
| SIRC FLJ11856 | 4 × 10³ | 6 | 150 |
| SIRC FLJ10060 | 2 × 10⁴ | 1 × 10³ | 3 × 10³ |
| SIRC PBaR-A2 | 6 × 10³ | Not tested | Not tested |

*BTI 3 is a molecular clone derived from 293 cells infected with PERV-A 14/220.

To investigate the expression of either of the human receptors in vivo a northern blot of human tissues was performed using standard methodologies. Briefly, northern blots of human tissues were purchased from Clontech Labs. and probed with a ³²P probe derived from PCR with the following primers: CCCAGTGGCAGGACAGTTG (SEQ ID NO: 18) and TCAGCGCGTTGGTGGC (SEQ ID NO: 19), using a FLJ11856 DNA template. The probe generated under these conditions will, even under stringent wash conditions (i.e., 0.1×SSC, 1% SDS at 55° C.) hybridize to RNA transcripts of FLJ 1856 and FLJ10060.

As shown in FIG. 2, expression can be detected in many cell types. Noteworthy is the expression of receptor in PBMC, strong expression in testes, and weak or absent expression in the bladder. These data underscore the need to ensure that PERV from a transplant is not passed sexually or to offspring.

In accordance with the present invention, there are provided herein processes for identification of cells susceptible to PERV infection. Such processes comprises screening of cells to determine their expression of the nucleotide sequence and therefore expression of a receptor. In human clinical trials it will enable companies performing PERV transmission monitoring, to specifically target cell that are infectable by PERV, thus increasing the sensitivity of screening assays and therefore the safety of xenotransplantation.

The present invention further provides a more precise determination of the likelihood of transmission of PERV beyond the xenograft recipient. In addition, identification of homologues present in other animal species and their associated expression profile facilitates more specific investigation of PERV infectivity in in vivo animal models.

In vitro expression of the protein thus enables non-susceptible cells to be rendered susceptible to infection, making them useful in determining the potential effects if PERV adapts to growth in the otherwise non-susceptible cell type X (for example, lung, sexual fluids, salivary glands, peripheral blood, and the like), the identification of co-factors required in addition to a receptor and that are needed to mediate infection.

In vivo expression of the protein (such as by transgenic animals) further enables non-susceptible animals (or animals with low susceptibility to infection) to be rendered susceptible to infection, thereby providing animal models for in vivo screening assays and other uses (and thus avoiding the need to use small primates and the like for such procedures). Of course, as will be readily recognized by those skilled in the art, the use of appropriately designed promoters with the constructs disclosed herein for expression of the receptor would make the expression tissue specific thereby facilitating the determination of the effects of infection on selected tissues (for example, infection of reproductive tissues and potential transmission to other organisms).

Identification of the protein that mediates infection also facilitates the development of anti-viral technologies, including screening for, or designing, highly specific agents that bind the receptor and block PERV entry agonistically/antagonistically. The present invention specifically contemplates that these can include both synthetic products as well as products of an immunization program. Additionally, identification of the region of the virus envelope that interacts with the receptor facilitates production of agents designed specifically for virus types.

The invention further relates to antibodies against the PERV receptors of the invention preferably monoclonal antibodies. The antibodies may be used to bind to the receptors to prevent or reduce PERV infection.

### EXAMPLE 2

### Generation of Monoclonal Antibodies Directed Against PERV Receptors

PERV-receptor cDNA clones in the pCDNA3 mammalian expression vector as described in Example 1 are introduced into mouse L-cells by electroporation using methods available for transformation of CHO cells (Barsoum, J. DNA and Cell Biology, 9:293 (1990)). Briefly, 5×10⁶ trypsinized cells are resuspended in 200 μl of 1×HeBS (20 mM HEPES buffer, pH 7.05, 137 mM NaCl, 5 mM KCl, 0.7 mM Na₂HPO₄, 6 mM dextrose) containing 50 μg of linearized plasmid and 50 μg sheared salmon testis DNA. Electroporations were performed using a GenePulsar apparatus (BioRad Laboratories, Hercules, Calif.) set at 290 V and 250 μFD for CHO cells or at 240 V and 250 μFD for L-cells, with cells chilled on ice prior to and for 10 minutes following electroporation. Cells were cultured for 48 to 72 hours prior to addition of G418 at 400 μg/ml to the culture medium. After the appearance of discrete colonies, the cells were trypsinized and replated to create single cell lines of L-cell transformants.

For mouse L-cell immunizations, 10⁶ transformed cells, rinsed and resuspended in PBS, are injected intraperitoneally into C3H mice. A boost is again given after 1-14 days with infusions following 5 days after a boost.

Hybridoma production follows standard protocols (see Current Protocols in Immunology, eds. J. E. Coligan et al., Wiley & Sons, New York, N.Y.). Splenocytes from immunized animals are fused to SP2/0-Ag14 myeloma cells and HAT selected culture wells tested for anti-receptor antibodies.

For hybridomas raised against transformed L-cells, culture wells are tested for antibody against transformed and untransformed CHO cells using standard flow cytometry procedures.

All references cited herein are incorporated by reference in their entirety for all purposes.

What is claimed is:

1. An isolated polynucleotide comprising a polynucleotide selected from the group consisting of:
(a) a polynucleotide at least 97% identical to the nucleotide sequence of SEQ ID NO: 15, and
(b) the full complement of (a).

2. The isolated polynucleotide of claim 1 wherein the polynucleotide of (a) is at least 99% identical to the sequence of SEQ ID NO: 15.

3. The isolated polynucleotide of claim 1 wherein the polynucleotide of (a) has the sequence of SEQ ID NO: 15.

4. An isolated polynucleotide comprising a polynucleotide selected from the group consisting of the sequence of SEQ ID NO: 11, SEQ ID NO: 13 and the full complement of either.

5. An isolated polypeptide comprising an amino acid sequence at least 96% identical to the amino acid sequence of SEQ ID NO: 16 wherein any differences in sequence are due only to conservative amino acid substitution.

6. The isolated polypeptide of claim 5 wherein said amino acid sequence is at least 98% identical to the amino acid sequence as SEQ ID NO: 16.

7. An isolated polypeptide comprising an amino acid sequence selected from the group consisting of SEQ ID NO: 12, 14, 16, 17 and immunogenic fragments thereof.

8. An antibody that reacts with a PERV-A receptor.

9. The antibody of claim 8 wherein said PERV-A receptor is selected from the group consisting of a human PERV-A receptor and a baboon PERV-A receptor.

10. The antibody of claim 8 wherein said antibody reacts with a polypeptide selected from the group consisting of SEQ ID NO: 10, 12, 14, 16 and 17 or an immunogenic fragment thereof.

11. The antibody of claim 8 wherein said antibody is a recombinant antibody.

12. A vector comprising a polynucleotide of claim 1, 2, 3, or 4.

13. A recombinant cell comprising the vector of claim 12.

14. A recombinant cell comprising a polynucleotide of claim 1, 2 3, or 4.

15. A recombinant mammalian cell engineered to express on its surface a receptor comprising the polypeptide of claim 5.

16. A recombinant mammalian cell engineered to express on its surface a receptor comprising the polypeptide of claim 6.

17. A recombinant mammalian cell engineered to express on its surface a receptor comprising the polypeptide of claim 7.

18. A process for identifying a compound that interferes with porcine endogenous retrovirus (PERV)-binding to a cell comprising:
(a) contacting a compound with a PERV-A receptor molecule under conditions promoting binding of said compound to said PERV-A receptor, and
(b) detecting binding of the compound to the PERV-A receptor,
thereby identifying a compound that interferes with PERV-binding to said cell.

19. The process of claim 18 wherein said PERV-A receptor comprises a polypeptide having an amino acid sequence selected from the group consisting of SEQ ID NO: 10, 12, 14, 16 and 17.

20. The process of claim 18 wherein said PERV-A receptor is part of a cell membrane.

21. The process of claim 20 wherein said cell membrane is part of an intact cell.

22. The process of claim 21 wherein said intact cell is a human cell.

23. The process of claim 21 wherein said intact cell is a recombinant cell engineered to express said polypeptide on its surface but that does not express said polypeptide absent said engineering.

24. The process of claim 23 wherein said cell has been transfected with a polynucleotide of claim 1, 2, 3, or 4.

25. The process of claim 18 wherein said contacting occurs in the presence of PERV and under conditions promoting binding of said PERV to said cell and detecting a decrease in binding of said PERV to said cell as compared to when said compound is not present thereby identifying a compound that interferes with PERV-binding.

26. A process for detecting the presence of a PERV-binding site on a cell comprising contacting a cell with an antibody of claim 8 and detecting binding of said antibody to said cell wherein said binding indicates the presence on said cell of a PERV binding site.

27. The process of claim 26 wherein said PERV-binding site is a PERV-A binding site.

28. A process for detecting the presence of a PERV-receptor gene in a cellular genome comprising contacting a sample of said genome with a probe comprising at least 15 contiguous nucleotides of a polynucleotide of claim 1, 2, 3, or 4.

29. The process of claim 28 wherein said fragment comprises at least 30 contiguous nucleotides.

30. The process of claim 28 wherein said fragment comprises at least 50 contiguous nucleotides.

31. The process of claim 28 wherein said comprises at least 100 contiguous nucleotides.

32. The process of claim 28 wherein said probe is a polynucleotide of claim 1, 2, 3, or 4.

33. A process for blocking a PERV receptor on a cell comprising contacting a cell expressing a PERV receptor with an agent that binds to said PERV receptor thereby blocking binding of PERV to said cell.

34. The process of claim 33 wherein said PERV receptor is PERV-A.

35. The process of claim 33 wherein said cell is a human cell.

36. The process of claim 33 wherein said agent is an antibody.

37. The process of claim 36 wherein said antibody is an antibody that reacts with a polypeptide comprising the amino acid sequence of SEQ ID NO: 10, 12, 14, or 16.

38. The process of claim 33 wherein said antibody is an antibody that reacts with an active fragment of claim a polypeptide comprising the amino acid sequence of SEQ ID NO: 10, 12, 14, or 16.

39. A process for protecting against PERV infection in a patient at risk of such infection comprising administering to said patient an effective amount of an agent that binds to PERV-A receptors thereby protecting against PERV infection.

40. The process of claim 39 wherein said PERV is PERV-A.

41. The process of claim 40 wherein the source of said PERV infection is a tissue used for transplantation.

42. The process of claim 41 wherein said transplantation is xenotransplantation.

43. The process of claim 39 wherein said agent is an antibody that reacts with a PERV-A receptor.

44. The process of claim 39 wherein said agent is an antibody that reacts with a polypeptide comprising the amino acid sequence of SEQ ID NO: 10, 12, 14, 16 or 17.

45. A method for producing a product comprising identifying an agent according to the process of claim 18 or 25 wherein said product is the data collected with respect to said agent as a result of said process and wherein said data is sufficient to convey the chemical structure and/or properties of said agent.

46. A transgenic animal comprising cells into whose genome has been inserted a polynucleotide encoding a PERV-A receptor and which cells express said receptor on their surface but wherein said animal does not express said receptor absent said insertion.

47. The transgenic animal of claim 46 wherein said polynucleotide encodes a polypeptide having the amino acid sequence of SEQ ID NO: 12, 14 or 16.

48. The transgenic animal of claim 46 wherein said polynucleotide is the polynucleotide of SEQ ID NO: 11, 13 or 15.

49. The transgenic animal of claim 46 wherein said animal is a mouse.

50. A process for identifying a compound that protects against PERV infection comprising administering to a transgenic animal of claim 46 a compound identified as interfering with PERV binding using the process of claim 18, then challenging said animal with a source of PERV and then determining that said animal does not exhibit the symptoms of PERV infection compared to when said compound has not been administered thereby identifying a compound that protects against PERV infection.

51. The process of claim 50 wherein said animal is a mouse.
